(19) United States
(12) Patent Application Publication
(10) Pub. No.: US 2009/0043199 A1
(43) Pub. Date: Feb. 12, 2009

(54) WIRELESS NETWORK HAVING PORTABLE ULTRASOUND DEVICES

(76) Inventors: Laurent PELISSIER, Vancouver (CA); Kris DICKIE, Chilliwack (CA); Kwun-Keat CHAN, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 12/188,193

(22) Filed: Aug. 7, 2008

Related U.S. Application Data
(60) Provisional application No. 60/955,331, filed on Aug. 10, 2007.

(51) Int. Cl. A61B 8/00

(52) U.S. Cl. 600/437

(57) ABSTRACT

A patient monitoring system has one or more ultrasound devices that monitor patients. The ultrasound devices can communicate to a central station by way of a wireless data communication network. Ultrasound images acquired by the ultrasound devices may be displayed at the central station. Alarms may be generated based upon conditions detected by the ultrasound devices. An ultrasound device may have a strap that permits it to be held in place with a transducer against the skin of a subject to permit ultrasound observation of the subject either continuously or intermittently over an extended period.

### CROSS-REFERENCE TO RELATED APPLICATION

This application claims priority from U.S. application No. 60/955,331 filed on 10 Aug. 2007 and entitled WIRELESS NETWORK HAVING PORTABLE ULTRASOUND DEVICES. This application claims the benefit under 35 U.S.C. §119 of U.S. application No. 60/955,331 filed on 10 Aug. 2007 and entitled WIRELESS NETWORK HAVING PORTABLE ULTRASOUND DEVICES which is hereby incorporated herein by reference.

### TECHNICAL FIELD

This invention relates to medical monitoring systems. The invention relates particularly to systems which apply ultrasound to detect physiological features or characteristics of a subject.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIG. 1 is a schematic view of a system according to an example embodiment of the invention.

FIGS. 2A and 2B show ultrasound devices having holding means such as straps.

FIGS. 3A and 3B show ultrasound devices deployed to monitor subjects for extended periods.

FIG. 4 is a schematic view showing an example embodiment of the invention deployed in a Intensive Care Unit.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The features as described herein may be combined in any suitable combinations with the features described in the commonly-owned US provisional patent applications entitled:
HAND-HELD ULTRASOUND SYSTEM HAVING STERILE ENCLOSURE (application No. 60/955327);
HAND-HELD ULTRASOUND IMAGING DEVICE HAVING RECONFIGURABLE USER INTERFACE (application No. 60/955,328);
POWER MANAGEMENT IN PORTABLE ULTRASOUND DEVICES (application No. 60/955,329);
HAND-HELD ULTRASOUND IMAGING DEVICE HAVING REMOVABLE TRANSDUCER ARRAYS (application No. 60/955,325); and
HANDHELD ULTRASOUND IMAGING SYSTEMS (application No. 60/977,353)
   all of which are hereby incorporated herein by reference. The features as described herein may also be combined in any suitable combinations with the features described in the commonly-owned US non-provisional patent applications which are filed on the same day as the instant application and entitled:
HAND-HELD ULTRASOUND SYSTEM HAVING STERILE ENCLOSURE (claiming priority from application No. 60/955,327);
HAND-HELD ULTRASOUND IMAGING DEVICE HAVING RECONFIGURABLE USER INTERFACE (claiming priority from application No. 60/955,328);
POWER MANAGEMENT IN PORTABLE ULTRASOUND DEVICES (claiming priority from application No. 60/955,329);
HAND-HELD ULTRASOUND IMAGING DEVICE HAVING REMOVABLE TRANSDUCER ARRAYS (claiming priority from application No. 60/955,325); and
HANDHELD ULTRASOUND IMAGING SYSTEMS (claiming priority from application No. 60/977,353)
   all of which are hereby incorporated herein by reference.

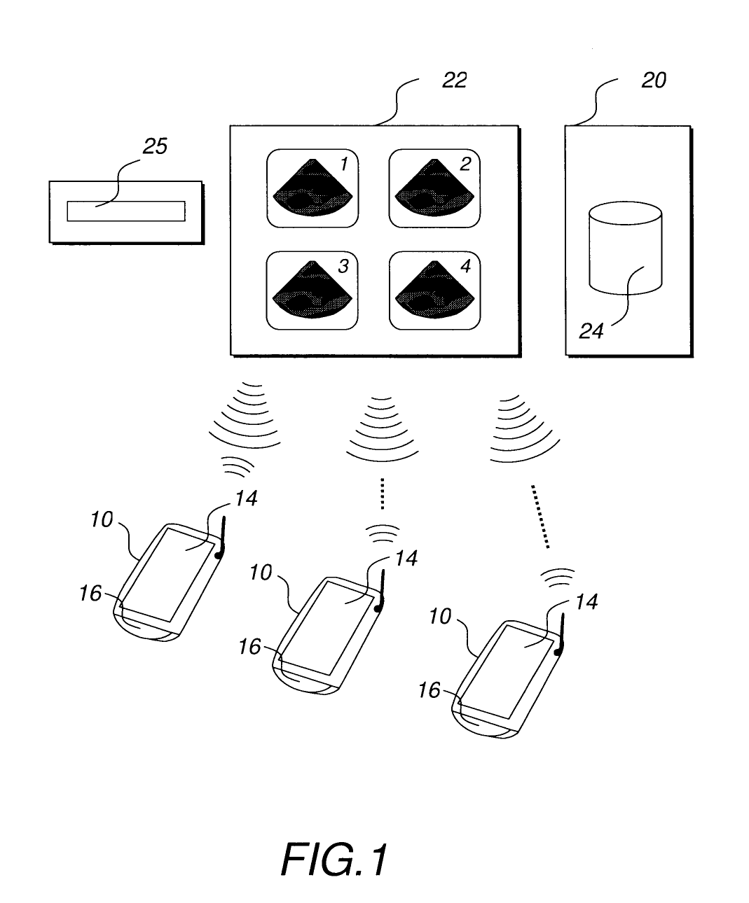

FIG. 1 is a schematic view of a network comprising a plurality of portable ultrasound devices 10 which are in wireless connection with a central station on the network. Communications between devices 10 and central station 20 may be provided by any wireless networking technology suitable for use in the area in which the network is deployed. The network may be deployed, for example, in a hospital, medical clinic, or the like. In some embodiments, the wireless networking may be provided by way of “WiFi” networking technology (e.g. a technology according to an IEEE 802.11 standard). In some embodiments, an interface to the wireless network is hard-wired into device 10. In other embodiments, device 10 has a slot that can receive a networking card. For example, a device 10 may have a slot for receiving a card such as a microSD card and a wireless interface that connects to the slot.

The protocol by which data is communicated between device 10 and central station 20 or other devices over the wireless network may optionally provide compression of data. This reduces the amount of data to be transmitted over the wireless network at the expense of some computation (and/or compression hardware) at the device 10.

Portable ultrasound devices 10 each have a transducer assembly 16 which can emit ultrasound signals. The ultrasound signals can interact with a subject and are reflected back to transducer assembly 16. Reflected signals are detected by device 10.

By applying suitable signal processing to reflected signals picked up at device 10, it is possible to generate an ultrasound image which contains information about the physiology of a subject or a physiological status of the subject. Different types of images or other information may be obtained by operating devices 10 in different modes. Signal processing algorithms are performed on reflected signals to generate the images or other desired information.

Device 10 may comprise a control and data processing system that controls the operation of device 10 and processes ultrasound data acquired at transducer assembly 16. The control and data processing system may comprise one or more programmed data processors, signal processing circuits, configurable logic circuits such as a field-programmable gate arrays, or a suitable combination thereof.

In a preferred embodiment, signal processing to generate ultrasound images is performed in each portable device 10 and the resulting images are displayed upon a screen 14 of the device 10. Image data from devices 10 is also, or in the alternative, conveyed back to central station 20 by way of the wireless network. At central station 20, the images may be displayed. It is not necessary for devices 10 to display ultrasound images in all embodiments.

In some embodiments, device 10 may generate alerts in response to detecting certain conditions relating to a subject being monitored. The alerts may be transmitted to central station 20 and may also, or in the alternative, be transmitted to portable devices being carried by medical personnel. For example, a device 10 may generate an alert upon a detected heart beat becoming irregular, exceeding or falling below threshold rates or a failure to detect a pulse signal. The alert may be transmitted to a portable device carried by a responsible physician and or a portable device carried by a responsible nurse. In some embodiments, central station 20 is configurable to forward alerts to one or more pagers or other portable devices that have been associated with a device 10.

In some embodiments, a user may carry a portable viewer which enables the user to view images from a device 10. The images may be received directly from the device 10 or may be retrieved from central station 20. Communication between the portable viewer and the device 10 and/or central station 20 may be provided by way of the wireless network. For example, a physician or other user may carry a portable viewer that has a user interface control which permits the physician or other user to select a device 10. Upon selecting the device 10 the viewer may receive an image originating from the device 10 and display the image for inspection by the physician or other user. In some embodiments, each device 10 has a code (which may be marked on the device 10) and the viewer may permit a user to enter the code to cause an image originating from the corresponding device 10 to be viewed.

In some embodiments, the signal processing performed at devices 10 is less intensive, but it is sufficient to allow an operator of the device 10 and to see an image that is clear enough to perform the desired imaging operation. The image data transmitted to central station 20 may be further processed at central station 20 to provide more refined images.

At central station 20 images may be displayed on a display 22 and/or stored in a database 24, printed on a hard copy unit 25 or transmitted to another destination by way of a computer network to which central station 20 is attached either wirelessly or by some other networking technology.

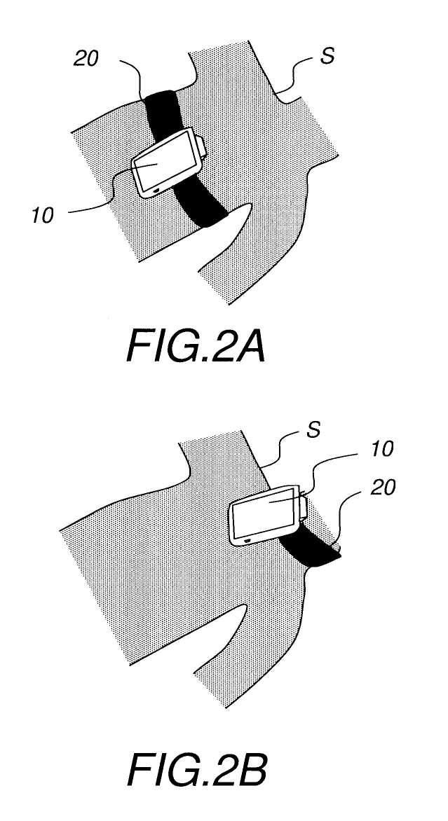

As shown in FIGS. 2A and 2B, a device 10 may be equipped with a strap 20, or other retaining mechanism, which allows the device 10 to be held in place with transducer assembly 16 pressed against the skin of a subject S. Device 10 may be held in place in such a location that an ultrasound image may be acquired of some anatomical feature subject S using the ultrasound device 10. When attached to a subject S, a device 10 may operate continuously or intermittently to generate an image of a structure within the subject S that is being monitored (for example, the image may be an image of a site of which the subject has been operated on, or a area within the body where fluids are building up or otherwise needs monitoring), or the operation of an organ within the subject S's body, such as the heart or the like. Device 10 may thus monitor subject S over an extended period of time.

In addition to or instead of generating an image, a ultrasound device 10 may acquire other information regarding the physiological status of subject S. For example, the device 10 may make Doppler blood flow measurements at certain points within the individual without necessarily acquiring an image.

Signals from devices 10 may be relayed back to central station 20 where images acquired by devices 10 may be viewed on display 22. Central station 20 may be configured to monitor features of the signals from devices 10 and to generate an alarm if those features deviate from a predicted pattern. For example, alarms may be generated upon one or more of the following events:
A device 10 ceases transmitting an image that can be recognized as representing some part of the anatomy of the subject S—this would occur if the device 10 somehow become dislodged from its desired position.
A device 10 detects that measured parameter, such as a Doppler blood flow parameter deviates from an acceptable value.
A device 10 detects the presence of a void within the subject S as might occur in the case that the subject was suffering from edema or internal bleeding. Signal processing algorithms applied at central station 20 to images generated by devices 10 can detect the possible presence of such voids in the images.

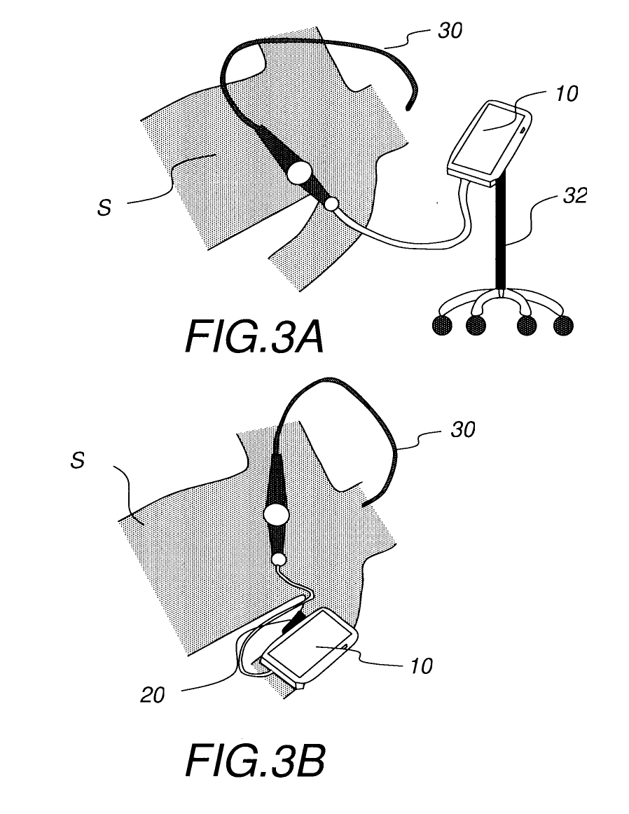

FIGS. 3A and 3B illustrate devices 10 equipped with trans-esophageal probes 30 may be inserted into subjects S by way of the esophagus (either through an opening or inserted into the subjects' mouths and down the subject's throats (if the subjects are unconscious or otherwise in a condition to receive a probe orally). Probes 30 include ultrasound transducers which transmit ultrasound signals and receive ultrasound signals that have been reflected from within the patient.

In FIG. 3A, device 10 is shown mounted on a stand 32. In FIG. 3B, device 10 is shown strapped to the subject's arm with a strap 20. In both embodiments, device 10 is a portable device which is mounted so that it can be used to monitor subject S over an extended period of time without the need that the device 10, or its probe 30, be continuously held or manipulated by a physician, nurse, or other operator.

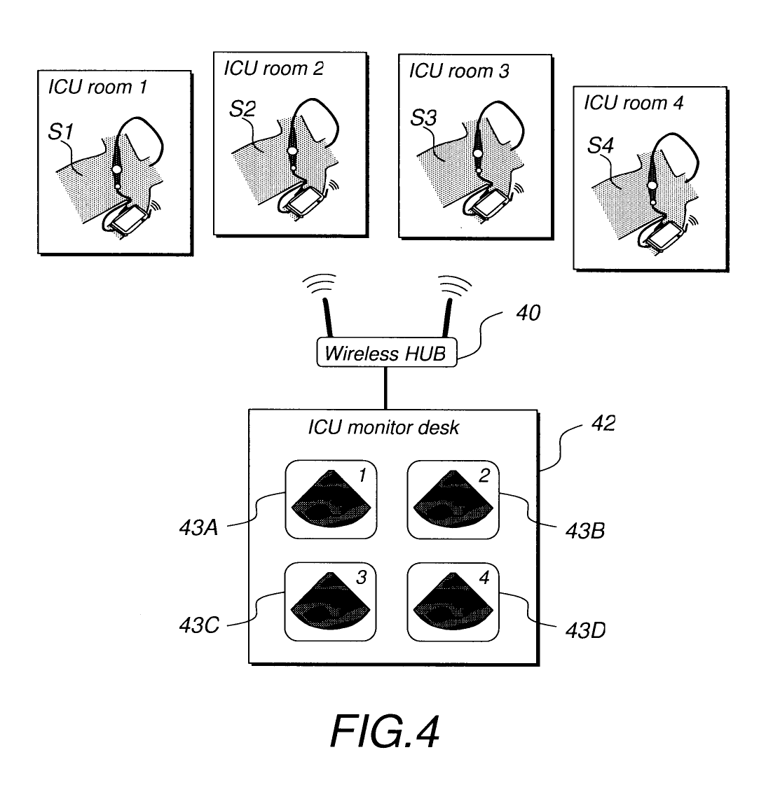

FIG. 4 shows an example embodiment of the invention deployed in a Intensive Care Unit. Subjects S1, S2, S3 and S4 are respectively located in Intensive Care Unit rooms 1, 2, 3 and 4. A device 10 is provided for monitoring each subject. Each device 10 has a trans-esophageal probe 30 which is inserted into the subject and monitors the functioning of the subject's heart. Each device 10 may operate in a Doppler mode, for example.

Signals from devices 10 are transmitted wirelessly to a wireless hub 40. Wireless hub 40 receives the signals and transmits the data from the signals to an ICU monitoring desk 42 which displays images 43A, 43B, 43C and 43D which correspond respectively to subjects S1, S2, S3 and S4.

By using the network shown in FIG. 4, a full ultrasound ICU type environment can be simulated at any desktop to monitor multiple or single patients.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments have been discussed above, those of skill in the art will recognize certain modifications, permutations, additions and sub-combinations thereof.

What is claimed is:

1. A patient monitoring system comprising:
an ultrasound device comprising an ultrasound transceiver, an ultrasound transducer connected to transmit and receive ultrasound signals, a control and data processing system configured to process ultrasound signals received at the ultrasound transducer and a wireless network interface; and,
a station connected to a network to receive ultrasound data from the ultrasound device, the station comprising a display and configured to display on the display information corresponding to the received ultrasound data,

2. A patient monitoring system according to claim 1 wherein the ultrasound device comprises a display and is configured to process the ultrasound signals to generate ultrasound image data and to display a first ultrasound image corresponding to the ultrasound image data on the display.

3. A patient monitoring system according to claim 2 wherein the ultrasound data comprises the ultrasound image data and station is configured to display on the display a second image corresponding to the ultrasound image data.

4. A patient monitoring system according to claim 3 wherein the station is configured to further process the ultrasound image data and the second image is refined relative to the first image.

5. A patient monitoring system according to claim 1 wherein processing the ultrasound signals comprises detecting a heart beat signal.

6. A patient monitoring system according to claim 5 wherein the ultrasound device comprises an elongated flexible esophageal probe wherein the ultrasound transducer is on the esophageal probe.

7. A patient monitoring system according to claim 5 wherein the ultrasound device is configured to generate an alarm signal in response to detecting that the heartbeat signal has: become irregular, has a rate exceeding a high threshold rate; has a rate below a low threshold rate; or is absent.

8. A patient monitoring system according to claim 3 wherein the station is configured to generate an alarm signal upon the station ceasing to receive the ultrasound image data from the device.

9. A patient monitoring system according to claim 3 wherein the station is configured to generate an alarm signal upon the ultrasound image data from the device ceasing to represent a selected anatomical structure of a patient being monitored.

10. A patient monitoring system according to claim 3 wherein the station is configured to generate an alarm signal upon detecting in the ultrasound image data the presence of a void within the subject.

11. A patient monitoring system according to claim 1 wherein the ultrasound device is configured to process the ultrasound signals to generate ultrasound image data and the system comprises a portable viewer, the portable viewer comprising a display, a wireless network interface, and a user control permitting selection of the ultrasound device,
wherein,
upon activation of the user control to select the device, the portable viewer is configured to receive the ultrasound image data from the ultrasound device and to display an ultrasound image corresponding to the ultrasound image data on the display.

12. A patient monitoring system according to claim 11 wherein the ultrasound device is marked with a code and the user control comprises a data entry mechanism permitting a user of the portable viewer to enter the code corresponding to the device.

13. A patient monitoring system according to claim 1 wherein the ultrasound device comprises a strap configured to permit the ultrasound device to be held in place with the transducer pressed against the skin of a subject.

14. A patient monitoring system according to claim 6 wherein the ultrasound device has a Doppler ultrasound mode.

15. A patient monitoring system according to claim 1 comprising a plurality of ultrasound devices wherein the station is connected to the network to receive ultrasound data from the plurality of ultrasound devices.

16. A patient monitoring system according to claim 1 wherein the ultrasound device is configured to generate ultrasound images continuously over an extended period of time.

17. A patient monitoring system according to claim 1 wherein the ultrasound device is configured to generate ultrasound images intermittently over an extended period of time.

18. An ultrasound device comprising:
a display,
an ultrasound transceiver,
an ultrasound transducer connected to transmit and receive ultrasound signals,
a wireless network interface,
a control and data processing system configured to: process ultrasound signals received at the ultrasound transducer to yield ultrasound image data; display an ultrasound image corresponding to the ultrasound image data on the display; and transmit the ultrasound image data by way of the wireless network interface, and
a strap configured to permit the ultrasound device to be held in place with the transducer pressed against the skin of a subject.

19. An ultrasound device according to claim 18 comprising an elongated flexible esophageal probe wherein the ultrasound transducer is on the esophageal probe.

20. An ultrasound device according to claim 19 configured to process the ultrasound signals to detect a heart beat signal.

21. An ultrasound device according to claim 20 wherein the ultrasound device is configured to generate an alarm signal in response to detecting that the heart beat signal has: become irregular, has a rate exceeding a high threshold rate; has a rate below a low threshold rate; or is absent.

22. An ultrasound device according to claim 18 marked with a code wherein the code permits communication with the ultrasound device by way of the wireless network interface.
